(19) United States
(12) Patent Application Publication
(10) Pub. No.: US 2009/0087473 A1
(43) Pub. Date: Apr. 2, 2009

(54) Osteoinductive bone graft material and manufacturing method

(76) Inventor: Hirohiko Inage, Tokyo (JP)

Correspondence Address: JACOBSON HOLMAN PLLC, 400 SEVENTH STREET N.W., SUITE 600, WASHINGTON, DC, 20004, US

(21) Appl. No.: 12/232,749

(22) Filed: Sep. 23, 2008

(30) Foreign Application Priority Data
Sep. 28, 2007 (JP) 253548/2007

(51) Int. Cl. A61K 9/00; A61K 38/16; A61P 19/00

(52) U.S. Cl. 424/423; 514/12

(57) ABSTRACT

In order to promote a bone regeneration, atelocollagen, collagen or metal to be utilized as a biomaterial is immersed in an organic solvent solution containing dissolved silanol polyhedral oligomeric silsesquioxane or silanol, then the immersed material is taken out of the solution and dried, and the dried silanol polyhedral oligomeric silsesquioxane or silanol is coated on the atelocollagen, collagen or metal, then the resultant compound is embedded in the bone defect.

### BACKGROUND OF THE INVENTION

1. Field of the Invention

The present invention relates to an osteoinductive bone graft material and a manufacturing method thereof, in which method atelocollagen, collagen or metal used to a biomaterial is immersed in an organic solvent solution containing solved silanol polyhedral oligomeric silsesquioxane ( hereinafter it is called silanol POSS in short), or silanol provided with the equal effect to that of the silanol POSS, in which silanol POSS silica is replaced by silanol, then the material is taken out of the solution and dried, the dried material of silanol POSS or silanol is coated on atelocollagen, collagen or metal, the resultant compound is embedded in bone defect in order to stimulate bone regeneration therein.

2. Description of the Prior Art

In treatment of bone fractures, tooth extraction socket and intensively resorbed bone, it is necessary to promote bone regeneration. In general, metal such as titanium inorganic biomaterial such as hydroxiapatite and extracellular matrix contained in bone has been employed as a biomaterial. It has been accepted that inorganic biomaterial is suitable at mechanical strength, and on the contrary, it causes immunoreaction, since it is not absorbed in vivo.

The osteoblast secretes bone matrix proteins and their cell membrane are tightly bound to the bone matrix protein. Since inorganic material such as titanium and gold which is not constituent of human body, osteoblast hard to connect directly on the surface of metal. It has been emphasized that it is difficult to synthesize new bone on the surface of inorganic biomaterial. The fact above has been pointed out as a problem in bone healing.

Bone matrix protein is consisting from collagen and non-collagenous protein such osteonectin and bone phosphoprotein. The collagen is estimated as 90% of bone extracellular matrix, elaborating a framework of bone matrix structure and playing an important role of mineralization of bone. Also, collagen has been use as a scaffold for bone regeneration in tissue engineering technology.

As described above, collagen has been used as a scaffold for generating bone because that it stimulates mineralization of newly formed bone. Japan Patent Laid-Open Official Gazette No. 107708/1995 (hereinafter, it called a document 1), J. bone Jt. surg (Am), 79-A, 495-502, 1997 (a document 2), and Biomaterials, 26, 5276-5284, 2005 (a document 3) describes that compound of collagen and inorganic material including in bone has been used as a scaffold for bone regeneration.

According to the descriptions shown in these documents, crystals of calcium and phosphate ions are precipitated on collagen fiber. That is, the document 1 describes collagen-phosphate compound, the document 2 a compound of hydroxiapatite or β-triphosphate calcium granules and collagen, and document 3 a nanohydroxyapatite crystallized collagen fiber. These bone graft substitute provide calcium and phosphate ions necessary for mineralization of collagen fiber. Furthermore, in order to give mechanical strength to the bone graft, composition of calcium and phosphate is given to the bone graft.

It is desirable that the bone graft having a purpose of bone regeneration of biomaterial has a quality of stimulating differentiation of osteoblast, protein synthesis and transport of inorganic substance, and being resolved in the new bone after it is formed. When only collagen is transplanted according to the prior art, immunoreaction is caused because it has high molecule degree. Due to that the conventional collagen has no function of induction of osteoblast differentiation, a long term is necessary to form new bone. As described above, according to the prior art using only collagen, several inconvenient and troublesome problems has been experienced when it is applied for clinics. Also the conventional bone graft of compound of collagen and inorganic material has a disadvantage of difficulty of resolving into the biomaterial.

There is no example that a compound of calcium and phosphate described in the documents 1 and 2 were induced calcification of bone. Hydroxyapatite described in the document 3 is disadvantageously difficult to resolve in tissue and finally remained in body as a foreign substance which cause immunoreaction. All compounds described in these documents fail to demonstrate induction of new bone formation or regeneration.

### SUMMARY OF THE INVENTION

The present invention has been accomplished so as to solve these problems above of the prior art. The present invention provides an osteoinductive bone graft material and a method for manufacturing the bone graft material, which method consists of the steps of immersing atelocollagen or collagen, which collagens being manufactured by cutting telepeptide of collagen to reduce immunoreactions, or metal used to biomaterial into an inorganic solvent solution, said solution containing silanol POSS-atelocollagen compound, silanol POSS-collagen compound, which compounds being formed by coating silanol POSS on the atelocollagen, collagen or metal, and containing silanol provided with an effect corresponding to that of silanol POSS-collagen compound, of taking out these substances of the solution, of drying these taken-out materials, of embedding silanol-atelocollagen compound, silanol-collagen compound, or silanol-metal compound, which compounds being formed by coating silanol on the atelocollagen, collagen or metal in order to promote bone regeneration.

The first object of the present invention is to provide a manufacturing method of bone graft material effecting on bone regeneration for human or biomaterial. The second object is to provide a bone graft material being able to use as a bone graft material when the amount of bone mass is decreased. The third object is a manufacturing method of the bone graft material or bone graft substitute being able to use as a bone graft for dental implants and artificial joints. These objects above and other objects, and novel characteristics of the present invention will be completely understood by reading the following detailed explanation with reference to the accompanying drawings. However, the drawings are exclusively for only explanation of the present invention, and no intension to restrict the scope of the present invention.

### BRIEF DESCRIPTION OF THE DRAWINGS

FIG. 1 is a perspective view of showing polyhedral oligomeric silsesquioxane.

FIG. 2 is a photograph of cultured cells on titanium discs. One being treated by immersing into an inorganic solvent solution having solved silanol POSS, and the other being not treated by immersing into the solution, both discs be cultured with osteoblast, indicating the result of comparative experiment and

FIG. 3 shows the result of rat bone regeneration experiments, respectively use silanol POSS-atelocollagen compound, or only atelocollagen.

### DETAILED EXPLANATION OF THE INVENTION

The present invention relates to an osteoinductive bone graft substitute having bone regenerating ability and a method for manufacturing thereof by the steps of immersing atelocollagen, collagen, or metal to be used to biomaterial, in an organic solvent solution containing solved silanol polyhedral oligomeric silasesquioxane or silanol POSS, or silanol provided with the equal effect to that of the silanol POSS, in which silanol POSS silica is replaced by silanol, taking the substance out of the solution and dried, coating the dried substance of silanol POSS or silanol on atelocollagen, collagen, or metal, embedding the resultant compound in bone defect in order to promote bone regeneration.

Next, the embodiment of the present invention will be explained in detail with reference to the accompanying drawings. According to the present invention, it is possible to employ silanol POSS, or silanol provided with a same function or effect as that of the silanol POSS as silanol provided with a same function or effect as that of the silanol POSS as a main material. Hereinafter, for convenience of explanation, the case or embodiment of the present invention in which silanol POSS is used as a main material will be described.

The main component for manufacturing osteoinductive bone graft substitute or bone graft material, and being use in this manufacturing method is polyhedral oligomeric silsesquioxane or POSS. The POSS has been developed and sold by several makers in the USA. According the present invention, it is not necessary to restrict the particular company, and preferably, it is recommend to use the POSSE which has been sold from 1998 with a trade name “POSS” by a company of Hybrid Plastics. Accordingly, the embodiment of the present invention will use the POSS manufactured by the company of Hybrid Plastics.

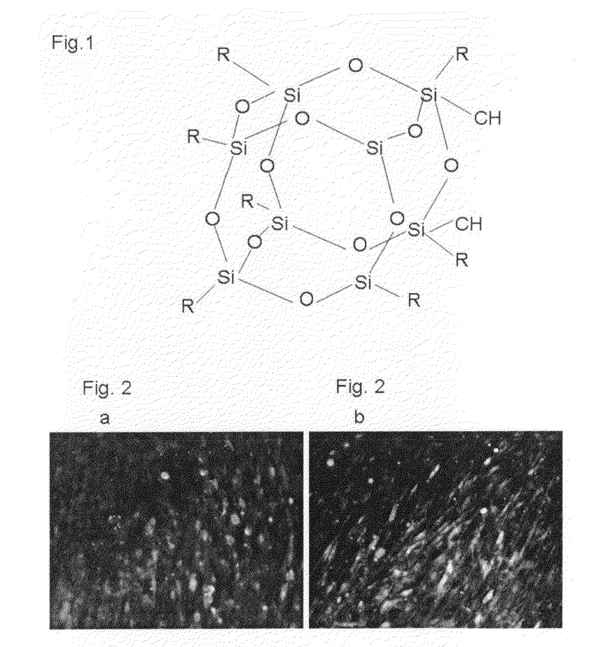

The POSS is a chemical compound of a diameter of approximately 1 nano-meter and of a tridimentional cage structure having a skeleton of Si—O—Si is shown in FIG. 1 of tridimentional structure figure. An oxygen portion of Si—O of a part of cage structure of POSS is connected to collagen and an exposed reactive group R is bonded with osteoblast.

Since, silicon of POSS is possible to be replaced by various substances, POSS is classified into alcohol POSS, phenol POSS and amine POSS etc.

POSS has characteristics of inorganic (silicon base) and organic substance (carbon base). Due to such hybrid structure of organic and inorganic, dramatic and excellent effects of improving mechanical characteristic, heat resistance property, optics characteristics, gas permeability, incombustibility, drug resistant characteristic, and ell adhesive property of the conventional polymer materials are obtained.

The inventor of the present invention has employed silanol POSS, in which a silicon of POSS is replaced by silanol (see US PAT. WO/2007/02236). The silanol employed to the present invention has a silanol base (Si—OH), has been developed and used as a biomaterial and has a characteristic of weak immunoreactions to biomaterial due to its low molecule property. A reactive group of a part of silanol POSS is connected to collagen and the remaining other reactive group is free and able to connect to fibroblast and osteoblast.

It is noted that titanium has been mostly used as a biomaterial for the treatment of the bone defects and bone fracture. Titanium also has been used as a biomaterial for dental implants and artificial joints. Therefore, the inventor of the present invention has carried out the following experiments in order to verify a cell adhesion property between silanol POSS and osteoblast.

The titanium discs respectively having 1 mm in thickness and 5 mm in diameter and various surface structures of 3 types such is shown below. The surface structure of the titanium discs are (1) machined surface, (2) acid etched surface by a solution of 50 volume % of sulfuric acid and 50 volume % of hydrogen fluoride, which are being mixed, and (3) laser treated surface by femto second laser.

According to the laser treatment way (3) above, repeating pulse of One Khz or of a high strength femto second light of wave length:800 mm generated from a titanium sapphire reproduction amplifier is applied to a titanium disc surface of a diameter:5 mm and a thickness:1 mm. The pulse used has a power of 200-800 μj per 1 pulse and a pulse width of 130 fs. The irradiation energy is controlled a half wavelength by means of an inclination beam splitter and the irradiation energy is concentrated on a surface of titanium disc by means of hiradeco cylindrical lens having a focal length of 80 mm. The test discs are scanned at a speed of 1 mm/second on the stage along the direction of X-Y.

The following experiments are carried out in order to demonstrate or inspect the function of silanol POSS. The titanium discs provided with their surface structure mentioned above are immersed into an organic solvent solution containing solved 0.1 to 4.5 volume % of silanol POSS and methylene chloride, then the titanium discs are taken out of the solution and dried so as to obtain the silanol POSS coated titanium discs. The bone stroma cell is isolated from rat bone marrow of precursor cell of osteoblast and it is cultured on the surfaces of silanol POSS coated titanium discs. In order to perform a comparison test, human cell line of osteoblast was cultured on the untreated titanium disc which disc is not immersed in the silanol POSS solution. After the cell culture described above are carried out on the titanium discs of 3 types, the number of cell on the titanium discs are counted at the first day, fifth day and seventh day after the cell culture.

The number of cultured cells on the machined titanium disc was 2.3 on the first day, 9 on the fifth day, and 10.2 on the seventh day of the culture in average. The number of cells on the acid etched surface was 33 on the first day, 38 on the fifth day, and 40 on the seventh day of the cell culture. On the titanium disc treated by femto second laser, they were 96 cells on the first day, 480 cells on the fifth day, and 1248 cells on the seventh day of the cell culture. On the contrary, on the machined titanium disc not treated with silanol POSS in the comparison test, they were 3.2 cells on the first day, 5 cells on the fifth day, and 9.7 cells on the seventh day of the cell culture.

In accordance with the experiments of the present invention, it was certified that the coating of silanol POSS certainly proliferates the precursor cell of osteoblast on the titanium discs. Furthermore, it was proved that the proliferation ratio or efficiency of cell on the titanium discs which are acid treated and laser treated is higher than that on the machined one. In particular, the highest efficiency of proliferation was attained on the titanium disc surface treated by laser.

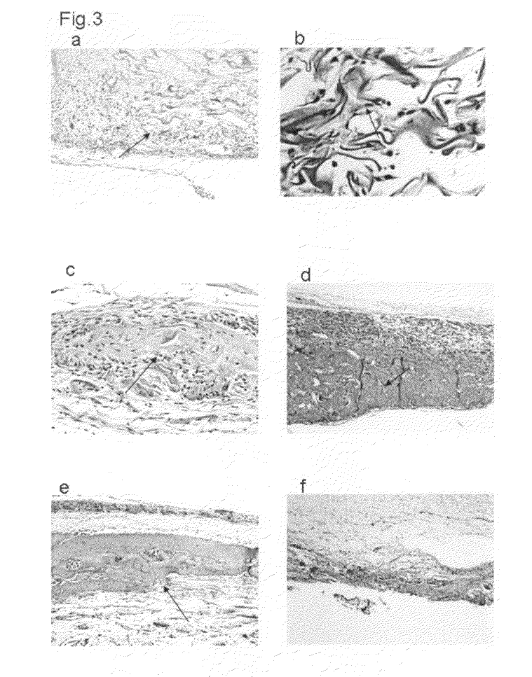

FIGS. 2a and 2b shows photographs of a fluorescent immunohistochemistry of the cell culture using osteoblast at the second week. FIG. 2a shows a photograph of fluorescent immunohistochemistry of the osteoblastic cell culture on the titanium disc surface not treated with silanol POSS and FIG. 2b shows same culture on silanol POSS coated titanium disc surface.

At the second week of the culture, much osteoblastic cell cultured on the titanium disc which is treated by silanol POSS express osteocalcin more than that of the collagen which is not treated by silanol POSS. Collagen is expressed at the early stage of osteoblastic cell differentiation, and osteocalcin is expressed at the late stage of matured osteoblast. This evidence clearly shows that silanol POSS promotes osteoblast differentiation. Each color shown in the photographs reveals the labeling as follows. Blue:nucleus of cell, Red:cell collagen is expressed therein, Green: cell osteocalcin is expressed therein, and Yellow:cell both collagen and osteocalcin are expressed therein.

It is certified from analyzing the photograph of FIG. 2a and 2b, and inspect the results of the cultures that to differentiation of cultured cell to osteoblast expressed on the titanium disc treated by silanol POSS higher than that on the titanium disc of non-treated.

The fact that the number and differentiation of osteoblast after the culture on the surface of silanol POSS coated titanium disc is high is suggests that it is due to the following phenomena. Silanol POSS has molecules of approximately 1 nano-meter in size and the tridimentional structure of free reactive group is suitably and tridimentionally adjusted to the surface structure of cell membrane of osteoblast, resulting in an anchoring effect and effective adhesion to osteoblast and in a function for providing an environment which is necessary to differentiate the adhered osteoblast.

Since silanol forms the skeleton of POSS, it is considered that a material coated with silanol in place of silanol POSS has the corresponding function with silanol POSS. Silanol has the identical silanol base with the frame work of silanol POSS cage, and a high possibility providing site to which cells are adhered. Accordingly, it is considered that the material prepared by a coating step of silanol on gold, gold alloy, nickel, and nickel alloy has the corresponding function to that of titanium provided with silanol POSS coated thereon. Also, it is expected that the corresponding substance having silanol base has the same effect.

Consequently, the inventor of the present invention has estimated that the compound prepared by coating of silanol POSS having the characteristics above on atelocollagen can be used to a purpose of bone regeneration. It is not necessary to restrict to the particular condensation of solution, however, preferably 0.1 to 4.5 volume %, mostly preferably 0.1 to 3.5 volume % of silanol POSS is solved in the organic solvent solution of ethanol or the like. According to the manufacturing method of the present invention, atelocollagen is immersed in the solution, the atelocollagen is taken out of the solution, then an excess amount of solution contained in the atelocollagen is absorbed by a filter paper, and atelocollagen is dried by air blowing and the like, resulting in a formation of silanol POSS-atelocollagen compound provided with atelocollagen coated by silanol POSS. It is noted that, when atelocollagen is immersed in an organic solvent solution which has methylene chloride or ethanol and the like and contains solved silanol POSS of 5 volume % and more than 5%, large crystals are formed on the atelocollagen after it is dried, causing immunoreaction, so that it is not possible to employ the process above to the manufacturing method of the present invention.

It is widely known that bone regeneration does not occur in calvaria. The inventor of the present invention found that it is possible to regenerate bone when silanol POSS is coated on atelocollagen forming a compound of both the materials above and the silanol POSS-atelocollagen compound is effectively used. The inventor has carried out an experiment of bone regeneration capability by embedding the compound in the bone defected portion of the rat calvaria. The process of the experiments is shown below.

The Wistar rats weighing 100 g are used in the experiment. Skin covering calvaria of the rat was cut under the anesthesia, exposing the top portion of calvarias. With droppings of a physiological solution of sodium chloride, a bone defect of a round shape of approximately 5 mm of the diameter was formed with a dental diamond disc at the calvaria. Atelocollagen of consisting from bovine type 1 and 3 of 0.002 g is immersed in the organic solvent solution containing methylene chloride and silanol POSS of 0.1 volume %, then the atelocollagen is taken out of the solution, excess amount of solvent is absorbed by a filter paper, the atelocollagen is air dried, silanol POSS is coated on atelocollagen, obtaining a compound of silanol POSS-atelocollagen. The compound is imbedded in the bone defect and a histological change of the bone defect was observed under the light microscope. During the experiment above, methylene chloride was used as an organic solvent solution. Ethanol may be used in place of methylene chloride. Other organic solvent solution having the same function as that of methylene chloride etc. can be used in the present invention.

FIG. 3 shows photographs indicating histological changes observed by a light microscope and a control experiment chronologically, respectively depicting the result of the experiment in which the compound was embedded in bone defects. On the fourth day of the experiment, connective tissue cells are accumulated around the silanol POSS-atelocollagen compound (see the arrow in the FIG. 3a). On the first week of the embodiment, the arrangement of embedded atelocollagen fiber becomes sparse and much precursor cells of osteoblast are observed between the space of the fiber (FIG. 3b and arrow). On the second week of the experimental embodiment, new bone (arrow) is formed at the central portion of bone defect (FIG. 3c). On the third week thereof, much amount of bone is filled in the bone defect portion (FIG. 3d). On the fourth week thereof, the spongy bone was completely replaced by the compact bone.

While, according to the control experiment in which only atelocollagen was embedded in the bone defect, only a small amount of spongy bone was formed around the bone defect even on the fourth week of embedment (FIG. 3f). In case that the compound was not embedded, no new bone was found entirely at the bone defect even by the fourth week of the embedment.

It was confirmed from the fact above that the silanol POSS-atelocollagen compound, which has a promotion effect of differentiation of osteoblast and synergy to atelocollagen becoming a source of bone formation, has a bone regeneration ability in rat calvaria, where no bone regeneration occurs. The experiment having the results above use in rats and it can be naturally estimated that the results will be occurred in both experiments using human.

In the experiment using rats, gel-type compound of silanol POSS-atelocollagen was used. When sol-type compound of silanol POSS-atelocollagen is embedded in bone defect, it is wormed by body temperature making it of gel-type compound, so that both the two types of sol and gel of compounds of silanol POSS and atelocollagen can be employed as a bone graft material having bone regeneration activity in the embodiment of the present invention.

Furthermore, the inventor has confirmed that the same effect as that described above is attained by making the silanol POSS-atelocollagen compound of sol type, which was beforehand prepared as one of gel type by means of neutral buffer liquid, and embedding the gel type silanol POSS-atelocollagen compound in rat bone defect.

It is well known that the ordinal collagen having telepeptide is a major constituent of bone matrix protein, and collagen is resorbed in osteoblast to synthesize again to form new collagen of bone matrix, resulting in a bone regeneration. Accordingly, atelocollagen and collagen of 4 to 30 types and of bovine having the characteristic mentioned above are used in place of the atelocollagen, preparing a compound of silanol POSS-collagen. It is estimated that the silanol POSS-collagen compound has the function same as that of the silanol POSS-atelocollagen compound. Also, it is estimated that the atelocollagen and collagen of the same type of human, pigs, rats, mice, rabbits, guinea pigs, sheep and goats, other mammals, fish and other invertebrates have the identical function to each other.

Also it is supposed that, when the silanol POSS-metal compound coated with silanol POSS is used on anyone of titanium, god, gold alloy, nickel, nickel alloy and the like to be used as a biomaterial in place of the silanol POSS-atelocollagen compound or silanol POSS-collagen compound, the silanol POSS-metal compound has approximately same function as that of silanol POSS-atelocollagen compound or silanol POSS-collagen compound.

Furthermore, also because that silanol forms a skeleton of POSS, it is supposed that it has the same function as that of silanol POSS. Consequently, it is supposed that the silanol-atelocollagen compound, silanol-collagen compound, or silanol-metal compound has the identical function to that of silanol POSS-atelocollagen compound, silanol POSS-collagen compound, or silanol POSS-metal compound.

In accordance with the present invention, silanol POSS-atelocollagen compound having two characteristics, which has an activity to connect to osteoblast forming bone and atelocollagen has low immunoreation by cutting the telopeptide of collagen is obtained. The present invention demonstrates that silanol POSS-atelocollagen compound shows regenerative activity in calvaria of rats, which does not occur bone regeneration, so that it is said that silanol POSS-atelocollagen compound has naturally a bone regeneration function for human. It is estimated that the silanol POSS-atelocollagen can be used for the treatment of the bone defects of human as a bone graft material when the amount of jaw bone and to like portion of human is reduced strongly, and to clinical application for orthopedics, dental treatment and bone treatment. Furthermore, it is possible to use the compound of the present invention as a bone graft material for dental implants and artificial joints of human.

In accordance with the present invention, silanol POSS-atelocollagen compound, which is prepared by the steps of coating silanol POSS provided with a adhesion activity to osteoblast forming bone tissue and a promotion function for differentiation of osteoblast on atelocollagen having low immunoreaction which is obtained by cutting the telepeptide of collagen of the main constituent of bone matrix, makes bone regeneration ability possible on the rat calvaria, which has been the particular one no bone regeneration is occurred. Consequently, the silanol POSS-atelocollagen compound naturally has bone regeneration ability for human, so it can be used to bone defects of human as a bone graft or bone graft material the bone amount of jaw bone and the like part of human is reduced or clinical application for orthopedics, dental treatment and bone treatment are able to do. Furthermore, it is possible to use the compound as a bone graft material for dental implants and artificial joints of human.

1. An osteoinductive bone graft material consisting of compound prepared by coating silanol polyhedral oligomeric silsesquioxane on atelocollagen.

2. An osteoinductive bone graft material consisting of compound prepared by coating silanol polyhedral oligomeric silsesquioxane on collagen.

3. An osteoinductive bone graft material consisting of a compound prepared by coating polyhedral oligomeric silsesquioxane on a metal used to biomaterial.

4. The osteoinductive bone graft material according to claim 1, in which silanol is used in place of the osteoinductive bone graft material.

5. The osteoinductive bone graft material according to claim 1, in which a bone matrix protein having a main substance of collagen is used in place of atelocollagen.

6. The osteoinductive bone graft material according to claim 1, in which atelocollagen or collagen is one of these of type 1 to 30.

7. A The osteoinductive bone graft material according to claim 1, in which atelocollagen or collagen has the identical type of fish, human, cows, pigs, rats, mice, rabbits, guinea pigs, sheep, goats and other invertebrates.

8. The osteoinductive bone graft material according to claim 3, wherein the metal used to biomaterial is one of titanium, gold, gold alloy, nickel, and nickel alloy.

9. A manufacturing method for an osteoinductive bone graft material, in which the compound is prepared by the steps of immersing the atelocollagen into an organic solvent solution having solved silanol polyhedral oligomeric silsequioxane of 0.1 to 4.5 volume %, taking out the atelocollagen of the solution, and drying the atelocollagen, and coating silanol polyhedral oligomeric silsequioxane on the atelocollagen.

10. A manufacturing method for an osteoinductive bone graft material, in which the compound is prepared by the steps of immersing the collagen into an organic solvent solution having solved silanol polyhedral oligomeric silsequixane of 0.1 to 4.5 volume %, taking out the collagen of the solution, and drying the collagen, and coating silanol polyhedral oligomeric silsequioxane on the collagen.

11. A manufacturing method of osteoinductive bone graft material, in which the silanol polyhedral oligomeric silsesquioxane-metal compound is prepared by the steps of immersing a metal used for biomaterial in an organic solvent solution consisting of solved silanol polyhedral oligomeric silsequioxane of 0.1 to 4.5 volume %, taking out the metal of the solution and drying the metal, and coating the silanol polyhedral oligomeric silseqioxane on the metal.

12. The manufacturing method of osteoinductive bone graft material according to claim 9, wherein silanol is used in place of silanol polyhedral oligomeric silequioxane.

13. The manufacturing method for osteoinductive bone graft material having bone regeneration function according to claim 9, wherein a bone matrix protein provide with its main substance of collagen is used in place of atelocollagen.

14. The manufacturing method of osteoinductive bone substitute having bone regeneration ability according to claim 9, wherein the atelocollagen or collagen is one of types 1 to 30.

15. The manufacturing method of osteoinductive bone substitute having bone regeneration ability according to claim 9, wherein atelocollagen or collagen is the atelocollagen or the collagen of the same type of human, bovine, sheep, pigs, rats, mice, rabbits, guinea pigs, goats, other mammals, fish, and other invertebrates.

16. The manufacturing method of osteoinductive bone substitute having bone regeneration ability according to claim 11, wherein the metal used to biomaterial is one of titanium, gold, gold alloy, nickel, and nickel alloy.
